(19) United States
(12) Patent Application Publication
(10) Pub. No.: US 2017/0027465 A1
(43) Pub. Date: Feb. 2, 2017

(54) SYSTEMS AND METHODS FOR CHARACTERIZING THE CONDUCTIVE PROPERTIES OF THE HEART

(71) Applicant: UNIVERSITY OF UTAH RESEARCH FOUNDATION, Salt Lake City, UT (US)

(72) Inventors: JOSH E. BLAUER, White Bear Township, MN (US); NASSIR F. MARROUCHE, Salt Lake City, UT (US); RAVI RANJAN, Salt Lake City, UT (US)

(21) Appl. No.: 15/225,471

(22) Filed: Aug. 1, 2016

Related U.S. Application Data
(60) Provisional application No. 62/199,813, filed on Jul. 31, 2015.

(51) Int. Cl. A61B 5/042; A61B 5/04; A61B 5/00

(52) U.S. Cl. CPC A61B 5/0422; A61B 5/6856; A61B 5/6858; A61B 5/04011; A61B 5/04012

(57) ABSTRACT

Electrophysiology mapping systems and methods that are used to determine longitudinal and transverse conduction velocities within myocardial tissue. The myocardial tissue can be contacted with at least three non-collinear electrodes. A selected electrical pacing protocol can include at least one pacing wave that is delivered to the at least three non-collinear electrodes. During each pacing wave, at least one pair of adjacent electrodes can generate an electrical activation pattern, and at least one additional electrode can detect the activation pattern. For each pacing wave, a processor determines a conduction velocity vector associated with the detection of the electrical activation pattern at a corresponding electrode. The processor can determine a singular value decomposition of the conduction velocity vectors determined during the electrical pacing protocol and use the singular value decomposition to determine the longitudinal and transverse conduction velocities within the myocardial tissue.

### CROSS-REFERENCE TO RELATED APPLICATION

This application claims priority to U.S. Provisional Application No. 62/199,813, filed Jul. 31, 2015. The disclosure of the above-referenced application is hereby incorporated herein by reference in its entirety.

### FIELD

This application relates to systems and methods for characterizing the conduction properties of the heart and, more particularly, for measuring conduction velocity and conduction anisotrophy.

### BACKGROUND

Many metrics of electrogram morphology used for substrate mapping are indirectly associated with mechanisms of arrhythmogenesis. For example, low voltage electrograms likely indicate scarred or otherwise abnormal tissues, but in many cases scarring can have an anti-arrhythmogenic affect, as seen with scarring created during ablation procedures. Conduction velocity (CV) is an electrophysiological measure that is directly related to arrhythmogenic mechanisms. However, in current clinical practice, there are no established techniques for robust characterization of CV properties. This inability to robustly characterize CV properties of the cardiac substrate is primarily attributable to the anisotropic nature of conduction in the heart. In other words, recording of sinus activation in the heart cannot elucidate the anisotropy of conduction. In experimental studies, conduction anisotropy is measured by pacing from dense electrode arrays or measurement systems from which the spread of activation can be captured. Although clinical systems are capable of pacing-based stimulation of the myocardium, current clinical multielectrode arrays lack sufficient spatial sampling density to fully characterize the spread of an activation impulse away from the pacing site.

Additionally, slowing of cardiac conduction velocity and associated changes in the anisotropy of conduction are important factors of the arrhythmogenic substrate. Currently, there are no techniques available, however, to estimate these parameters in a quick and reliable manner.

Thus, there is a need for systems and methods that facilitate robust CV property characterization using a repetitious stimulation and recording protocol. There is a further need for systems and methods for normalization of CV measures from multiple stimulus sites to account for variations in the CV properties that occur as an activation impulse moves away from a stimulus site. There is also a need for systems and methods for measuring CV and conduction anisotrophy, without the requirement of activation time assignment.

### SUMMARY

In one aspect, disclosed are systems and methods for activation pattern permutation (APP) mapping that provides computed tomography of cardiac conduction properties. The conduction velocity (CV) of electrical impulses through cardiac tissues is an important factor in the development of arrhythmia. CV along muscle fibers can progress faster than across fibers. Consequently, cardiac tissues can exhibit longitudinal (along the length of the fiber) and transverse (across the fiber) CVs at any given point. These CVs are known to change with various disease processes and can provide an indication of diseased tissue. In clinical electrophysicology, there are currently no techniques for detecting and discriminating between the longitudinal and transverse aspects of CV. Disclosed herein are systems and methods for pacing, recording and analysis that extract longitudinal and transverse conduction velocities. Optionally, these longitudinal and transverse conduction velocities can be extracted using a catheter, such as a standard clinical loop catheter.

In another aspect, disclosed herein is an electrophysiology mapping method. The method can include contacting myocardial tissue with at least three non-collinear electrodes of a plurality of electrodes, wherein the at least three non-collinear electrodes concurrently contact the myocardial tissue. The method can also include using a pulse generator to deliver a selected electrical pacing protocol to the at least three non-collinear electrodes, wherein the selected electrical pacing protocol comprises at least one pacing wave. The method can further include, during each pacing wave of the selected electrical pacing protocol, generating an electrical activation pattern using at least one pair of adjacent electrodes of the at least three non-collinear electrodes and using at least one additional electrode of the plurality of electrodes to detect the activation pattern. The method can further include, for each pacing wave, using a processor in communication with the plurality of electrodes and the pulse generator to determine a conduction velocity vector associated with the detection of the electrical activation pattern at a corresponding electrode. The method can still further include using the processor to determine a singular value decomposition of the conduction velocity vectors determined during the electrical pacing protocol. The method can still further include determining longitudinal and transverse conduction velocities within the myocardial tissue based upon the singular value decomposition of the conduction velocity vectors.

In an additional aspect, disclosed herein is an electrophysiology mapping system having a plurality of electrodes, a pulse generator, and a processor. At least three of the electrodes are non-collinear and configured to simultaneously contact myocardial tissue within a heart of a subject. The pulse generator can be electrically coupled to the electrodes, and the processor can be communicatively coupled to the pulse generator and the electrodes. The pulse generator can be configured to deliver a selected electrical pacing protocol to the electrodes. The selected electrical pacing protocol can comprise at least one pacing wave. During each pacing wave of the selected electrical pacing protocol, at least one pair of adjacent electrodes can be configured to generate an electrical activation pattern and at least one additional electrode can be configured to detect the activation pattern. For each pacing wave, the processor can be configured to determine a conduction velocity vector associated with the detection of the electrical activation pattern at a corresponding electrode. The processor can be configured to determine a singular value decomposition of the conduction velocity vectors determined during the electrical pacing protocol. The processor can be further configured to determine longitudinal and transverse conduction velocities based upon the singular value decomposition of the conduction velocity vectors.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

These and other features of the preferred embodiments of the invention will become more apparent in the detailed description in which reference is made to the appended drawings wherein:

FIG. 1 shows an anisotropic conductivity model of excitation propagation initiated by look catheter pacing. Extracellular potential of this model was shown 10 ms after the start of pacing from the white hexagonal region. The purple hexagons indicate the position of recording electrodes. Further, the black arrows indicate the fiber orientation of the model.

FIG. 2 shows the characterization of local conduction properties with a loop catheter. As shown in the left column, vectors at recording electrodes (purple spheres) point away from a pacing site. The length and color correspond to the CV. The center column shows that the CV vectors are normalized to a fixed distance from the pacing site. The right column is a compact representation of regional conduction.

FIG. 3A-FIG. 3C show the linear regression of CV versus the distance of the recording electrode from the pacing site. FIG. 3A and FIG. 3B show the slope for the anisotropic and isotropic computational models, respectively, and FIG. 3C shows the in vivo model, all of which can be used to normalize all CV measurements to 20 mm from the pacing site.

FIG. 4 is a compact representation of inter-loop conduction properties of a right atrium at two sites. Both sites were interrogated with multiple activation patterns by bipolar pacing between electrodes around the loop. The normalized CV vectors shown indicate the direction and velocity of conduction.

FIG. 5A-FIG. 5C depict representative unipolar, Laplacian and mean Laplacian electrograms (MLE). FIG. 5A illustrates an exemplary MLE recorded from the ventricles of a guinea pig heart during three paced beats. FIG. 5B provides an expanded timescale comparison of the unipolar, Laplacian and MLE that correspond to the boxed region of FIG. 5A. FIG. 5C illustrates the quantification of the temporal location of the peaks in the MLE.

FIG. 6A-FIG. 6D show correlation of conduction velocity and anisotropy with features of the MLE. FIG. 6A shows representative activation time isochrone maps recorded from a guinea pig heart under control or flecainide conditions. The closer spacing of isochronal lines with flecainide demonstrates slowing of conduction. FIG. 6B shows MLEs corresponding to the maps of FIG. 6A. FIG. 6B shows that the temporal locations of both peaks are shifted to the right, as indicated by the shift in TP1 and TP2. FIG. 6C is a scatter plot of CV calculated from the maps of FIG. 6A versus TP1 (for longitudinal CV) and TP2 (for transverse CV). FIG. 6D is a scatter plot of anisotropy based on CV versus anisotropy based on TP. The corresponding correlation coefficients and confidence intervals (“CI”) are indicated. It is contemplated that the regression lines (ordinary least squares regression) shown in FIGS. 6C and 6D are for illustrative purposes only.

FIG. 7A-FIG. 7D are graphs showing paired comparisons of: 1) longitudinal CV (FIG. 7A); 2) transverse CV (FIG. 7B); 3) TP1 (FIG. 7C); and 4) TP2 (FIG. 7D), under controlled conditions and under conditions of pharmacologically-induced conduction slowing. P-values from Welch's t-test are indicated.

FIG. 8A-FIG. 8C illustrate reconstruction of the MLE in a bi-domain simulation of conduction. FIG. 8A provides representative simulated activation time maps and corresponding MLEs with the 8×8 recording array rotated at different angles with respect to fiber orientation. Also shown is the fractionation of the MLE as the array is rotated 45 degrees. FIG. 8B is a matrix representation of the 3×3 coefficient stencil used to calculate the Laplacian electrogram for each of the 36 central electrodes. FIG. 8C is a matrix representation of the equivalent 8×8 coefficient stencil of the MLE. FIG. 8C depicts the resulting cancellation of the central coefficients and the heterogenous weight of the corner coefficients.

FIG. 9A and FIG. 9B provide representations of the revised rotationally independent recording configuration. FIG. 9A shows recording electrodes organized in two concentric rings of different diameters, referred to as a Dual Ring Array (“DRA”). Analogous to the coefficient stencil shown in FIG. 8C, the electrodes on the inner ring have negative coefficients (−1) and the electrodes on the outer ring have positive coefficients (+1). FIG. 9B illustrates a fixed diameter recording array with the inner and outer electrodes placed on the same ring.

FIG. 10A-FIG. 10E show the simulation of MLEs based on the DRA. FIG. 10A shows a simulated activation time map with the position of the DRA electrodes indicated. FIG. 10B is a graph showing the MLE corresponding to the map of FIG. 10A. The temporal location of the first and second peaks and the zero crossing are shown. FIG. 10C-FIG. 10E are isopotential maps corresponding to the time points of the first peak (FIG. 10C), the second peak (FIG. 10D), and the zero crossing (FIG. 10E). The arrows point to the electrodes closest to the propagating wavefront at each time point. The pacing site is indicated as a larger dot in the center of the maps of FIG. 10C-FIG. 10E.

FIG. 11A-FIG. 11D provide a comparison of CV and anisotropy estimates for the DRA-based MLE and activation time map methods. FIG. 11A is a plot of correlation coefficients describing the relationship between CV calculated from activation time maps versus different features of the MLE. The dotted line indicates r=0.95. P1 represents the first peak, P2 represents the second peak, and ZC represents the zero crossing. FIG. 11B-FIG. 11D are plots of the relationship between CV calculated from activation time maps versus CV calculated from the MLE for: 1) longitudinal CV (FIG. 11B); 2) transverse CV (FIG. 11C); and 3) conduction anisotropy (FIG. 11D). The solid lines represent the type II (major axis) regression line. The line slope and the corresponding CI are indicated for each regression.

FIGS. 12A-12B are schematic illustrations of an exemplary diagnostic catheter capable of acquiring near-field electrograms (“EGMs”). As shown, the catheter can have a basket-type configuration containing a central reference electrode surrounded by splines. The splines can contain multiple exploratory electrodes that allow for clinical mapping with near-field EGMs. FIG. 12A depicts the splines of the catheter in a radially retracted condition, whereas FIG. 12B depicts the splines of the catheter in a radially expanded condition.

FIG. 13A and FIG. 13B depict singular value decomposition (“SVD”) of CV vectors. FIG. 13A shows CV vectors acquired with multiple activation pattern mapping protocol that have been normalized for distance from the pacing site and projected to the center of the loop array. FIG. 13B shows the first two principal axes recovered from SVD of CV vectors, indicating the direction of longitudinal and transverse conduction (red and blue cones, respectively.)

FIG. 14 is a side perspective view of an exemplary loop catheter that supports a plurality of pacing and/or recording electrodes as disclosed herein.

FIG. 15 is a schematic diagram depicting the interaction between the electrodes, the pulse generator, and the processor as disclosed herein.

The patent or application file contains at least one drawing executed in color. Copies of this patent or patent application publication with color drawing(s) will be provided by the Office upon request and payment of the necessary fee.

### DETAILED DESCRIPTION

The present invention can be understood more readily by reference to the following detailed description and appendix, which include examples, drawings, and claims. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a catheter” can include two or more such catheters unless the context indicates otherwise. Similarly, reference to “an electrode” can include two or more such electrodes unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, the term “subject” refers to both human and animal subjects.

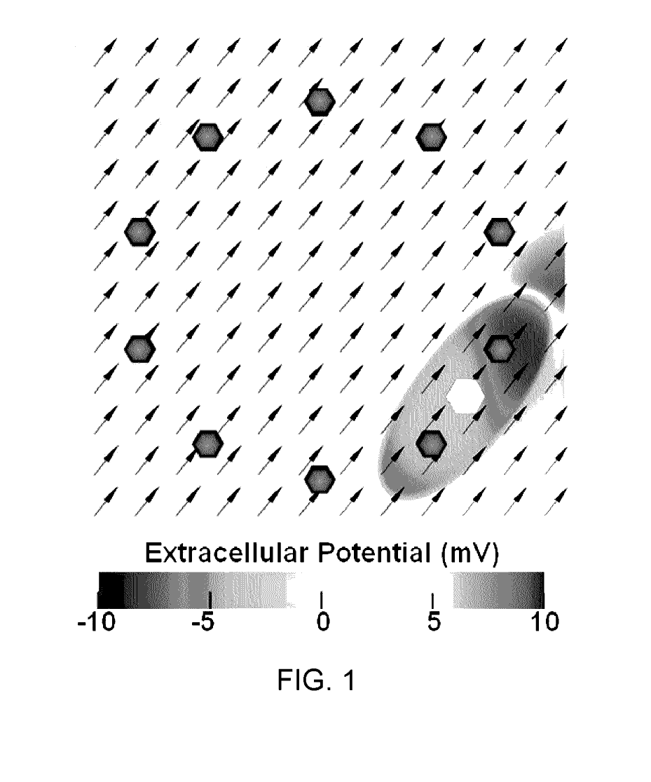

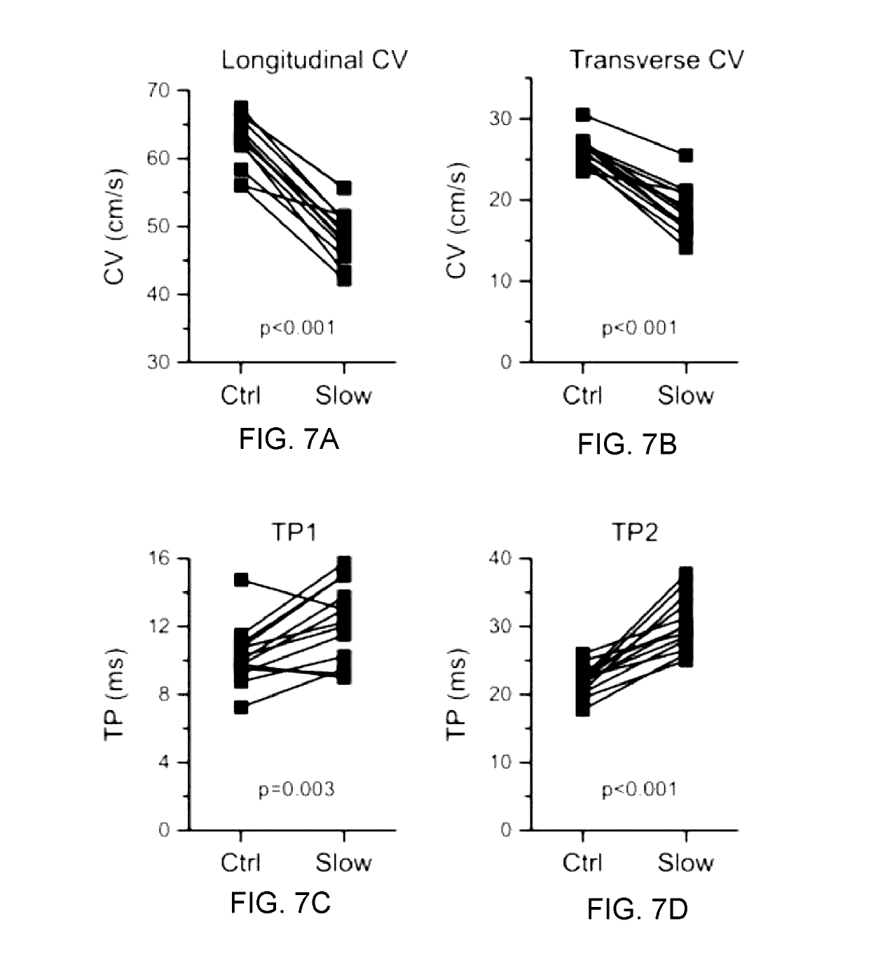

Disclosed herein, in various aspects and with reference to FIGS. 1-15, are systems and methods for characterizing the conductive properties of the heart.

In one aspect, disclosed are systems and methods for activation pattern permutation (APP) mapping that provides computed tomography of cardiac conduction properties. The conduction velocity (CV) of electrical impulses through cardiac tissues is an important factor in the development of arrhythmia. CV along muscle fibers can progress faster than across fibers. Consequently, cardiac tissues can exhibit longitudinal (along the length of the fiber) and transverse (across the fiber) CVs at any given point. These CVs are known to change with various disease processes and can provide an indication of diseased tissue. In clinical electrophysicology, there are currently no techniques for detecting and discriminating between the longitudinal and transverse aspects of CV. Disclosed herein are systems and methods for pacing, recording and analysis that extract longitudinal and transverse conduction velocities. Optionally, these longitudinal and transverse conduction velocities can be extracted using a standard clinical loop catheter.

In one aspect, disclosed herein are systems and methods for measuring cardiac tissue properties (e.g., conduction properties). In another aspect, disclosed herein are systems and methods for treatment of cardiac arrhythmias. In an additional aspect, the systems and methods disclosed herein can require no new hardware. In a further aspect, the systems and methods can permit active interrogation, rather than passive interrogation, of tissue health. In another aspect, the systems and methods can confirm ablation line continuity. In another aspect, the systems and methods can identify diseased regions.

In another aspect, disclosed herein are methods of simulation of the myocardium at multiple non-collinear electrode locations on a transvenous electrophysiological recording array that can elicit potential waves that propagate through the myocardium with variable conduction velocities. It is contemplated that the variability of the wave velocity can be a function of the local tissue architecture including fiber orientation and the presence of fibrosis or scar. In a further aspect, the systems and methods disclosed herein can measure temporal conduction of the action potential through the myocardium by pacing from any adjacent set of electrodes, and simultaneously recording the electrograms of the resultant action potential from the remaining electrodes on the recording array. In this aspect, subsequent pacing and recording from the remaining combinations of electrode pairs can then permit tomographic reconstruction of the local tissue anisotropy.

### Electrophysiology Mapping Methods

In exemplary aspects, disclosed herein is an electrophysiology mapping method. In one aspect, the method can comprise contacting myocardial tissue with at least three non-collinear electrodes of a plurality of electrodes. In this aspect, the at least three non-collinear electrodes can be configured to concurrently contact the myocardial tissue. In another aspect, the method can comprise using a pulse generator to deliver a selected electrical pacing protocol to the at least three non-collinear electrodes. In this aspect, the selected electrical pacing protocol can comprise at least one pacing wave. In an additional aspect, the method can further comprise, during each pacing wave of the selected electrical pacing protocol, generating an electrical activation pattern using at least one pair of adjacent electrodes of the at least three non-collinear electrodes and using at least one additional electrode of the plurality of electrodes to detect the activation pattern. In a further aspect, the method can comprise, for each pacing wave, using a processor in communication with the plurality of electrodes and the pulse generator to determine a conduction velocity vector associated with the detection of the electrical activation pattern at a corresponding electrode. In still a further aspect, the method can comprise using the processor to determine a singular value decomposition of the conduction velocity vectors determined during the electrical pacing protocol. In yet another aspect, the method can further comprise determining longitudinal and transverse conduction velocities within the myocardial tissue based upon the singular value decomposition of the conduction velocity vectors.

In further exemplary aspects, the method can further comprise, during each pacing wave of the selected electrical pacing protocol: using the at least one additional electrode to record an electrogram; using the processor to determine an activation time of a corresponding recording electrode based upon each recorded electrogram; and using the processor to determine the conduction velocity vector associated with the corresponding recording electrode based upon the determined activation time and a known distance between a pacing electrode and the corresponding recording electrode.

Optionally, in additional aspects, the at least one pacing wave can comprise a plurality of pacing waves. In these aspects, during each pacing wave, a single pair of adjacent electrodes can generate a corresponding electrical activation pattern through the myocardial tissue. In exemplary aspects, each pacing wave can be a depolarization wave.

In further exemplary aspects, it is contemplated that each pacing wave can have desired parameters, including, for example and without limitation, a selected cycle length, a selected amplitude, a selected pulse width, and the like. In one aspect, it is contemplated that each pacing wave can have a cycle length ranging from about 100 ms to about 1 s. In another aspect, it is contemplated that each pacing wave can have an amplitude ranging from about 5 mA to about 15 mA. In a further aspect, it is contemplated that each pacing wave can have a pulse width ranging from about 1 ms to about 3 ms. Although exemplary parameter values are disclosed herein, it is contemplated that any selected parameters can be employed to achieve a particular result within myocardial tissue.

In another aspect, the at least one additional electrode that detects the electrical activation pattern can comprise each of the electrodes of the at least three non-collinear electrodes that is not used to generate the electrical activation pattern. In this aspect, it is contemplated that each of the at least three non-collinear electrodes can serve as a pacing electrode during at least one pacing wave and as a recording electrode during at least one pacing wave. It is further contemplated that the at least one additional electrode that detects the electrical activation pattern can further comprise at least one reference electrode that does not contact the myocardial tissue.

In another aspect, the at least three non-collinear electrodes can be supported on a catheter. In this aspect, the method can comprise positioning the catheter within a heart of a subject to contact myocardial tissue within the heart with the at least three non-collinear electrodes. Optionally, in one exemplary aspect and as further disclosed herein, the catheter can be a loop catheter having a loop portion. In this aspect, it is contemplated that the at least three non-collinear electrodes can be secured to the loop portion of the catheter.

Optionally, in another exemplary aspect and as further disclosed herein, the catheter can be a basket electrode catheter having a longitudinal axis and a plurality of splines. In this aspect, each of the at least three non-collinear electrodes can be secured to a spline of the basket electrode catheter, and the plurality of splines of the catheter can be selectively deformable about and between a radially retracted condition and a radially expanded condition. In the radially expanded condition, it is contemplated that the splines can be compressed such that an intermediate portion of each spline extends radially outwardly relative to the longitudinal axis of the catheter. In these aspects, the method can comprise positioning the splines of the catheter in the radially expanded condition to contact myocardial tissue within the heart with the at least three non-collinear electrodes. Optionally, in additional aspects and as further disclosed herein, the plurality of electrodes can further comprise a central reference electrode positioned within a central area defined by the plurality of splines of the catheter. In these aspects, when the plurality of splines are in the radially expanded condition, the central reference electrode can be equidistant from the at least three non-collinear electrodes.

### Electrophysiology Mapping Systems

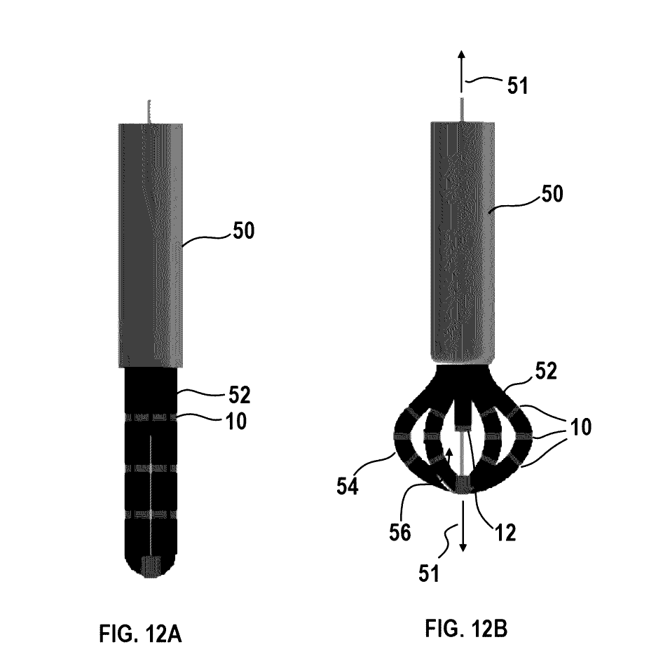

In an additional aspect, with reference to FIGS. 12A-12B and 14-15, disclosed herein is an electrophysiology mapping system 100 having a plurality of electrodes, a pulse generator 20, and a processor 30. At least three of the electrodes 10 are non-collinear and configured to simultaneously contact myocardial tissue within a heart of a subject. As schematically depicted in FIG. 15, the pulse generator 20 can be electrically coupled to the electrodes, and the processor 30 can be communicatively coupled (e.g., through a wired or wireless connection) to the pulse generator 20 and the electrodes 10. The pulse generator 20 can be configured to deliver a selected electrical pacing protocol to the electrodes. Optionally, the pulse generator 20 can be provided as a component of an electrophysiology recording (pacing and mapping) system as is known in the art. However, it is contemplated that any conventional electrical pulse generator configured for use in cardiac applications can be used.

In exemplary aspects, the selected electrical pacing protocol can comprise at least one pacing wave. During each pacing wave of the selected electrical pacing protocol, at least one pair of adjacent electrodes can be configured to generate an electrical activation pattern and at least one additional electrode can be configured to detect the activation pattern. For each pacing wave, the processor 30 can be configured to determine a conduction velocity vector associated with the detection of the electrical activation pattern at a corresponding electrode. In an additional aspect, the processor 30 can be configured to determine a singular value decomposition of the conduction velocity vectors determined during the electrical pacing protocol. In a further aspect, as further disclosed herein, the processor 30 can be further configured to determine longitudinal and transverse conduction velocities based upon the singular value decomposition of the conduction velocity vectors.

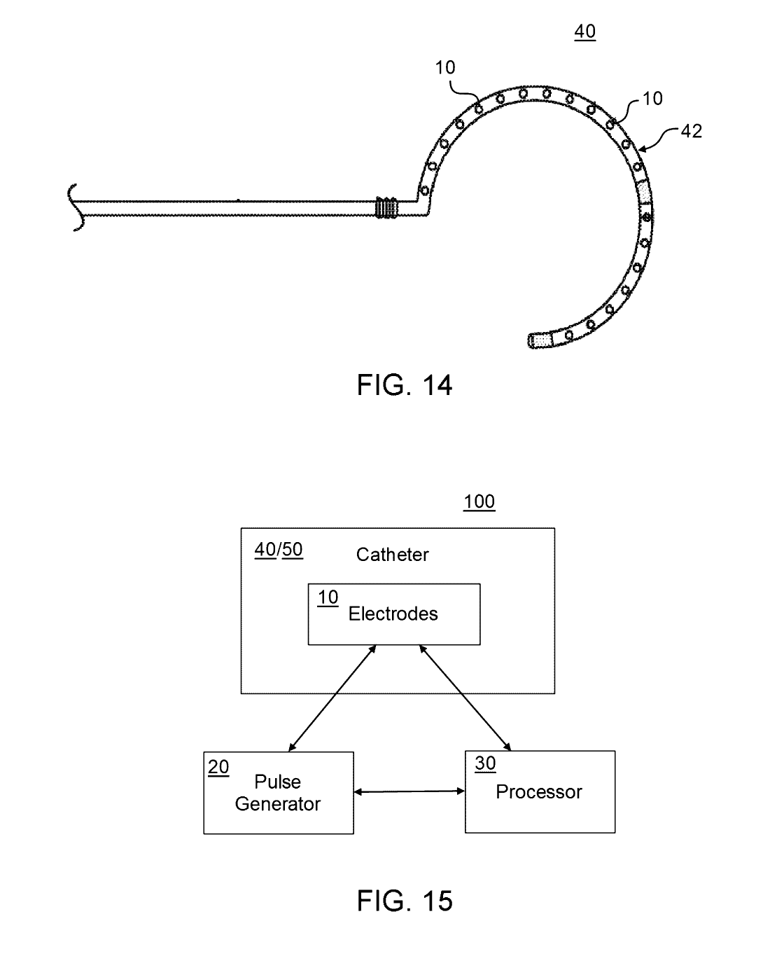

In exemplary aspects, the system 100 can further comprise a catheter that supports the plurality of electrodes 10. Optionally, in some aspects, and as further disclosed herein and shown in FIG. 14, it is contemplated that the catheter can be a loop catheter 40 having a loop portion 42 as is known in the art. In these aspects, the at least three non-collinear electrodes can be secured to the loop portion 42 of the catheter 40 in a conventional manner. In exemplary aspects, the at least three electrodes can be circumferentially spaced relative to a loop or arcuate shape defined by the loop portion 42 of the catheter 40 as further disclosed herein. Exemplary loop catheters include, for example and without limitation, the LASSO®NAV catheter (Biosense Webster, Inc.).

Alternatively, in other optional aspects, and as further disclosed herein and shown in FIGS. 12A-12B, the catheter can be a basket electrode catheter 50 having a plurality of splines 52. In these aspects, it is contemplated that each of the at least three non-collinear electrodes can be secured to a spline 52 of the basket electrode catheter 50. In further exemplary aspects, the catheter 50 can have a longitudinal axis 51, and the plurality of splines 52 of the catheter can be selectively deformable about and between a radially retracted condition and a radially expanded condition. In the radially expanded condition, the splines 52 are compressed such that an intermediate portion 54 of each spline extends radially outwardly relative to the longitudinal axis 52 of the catheter 50. In exemplary aspects, it is contemplated that the splines can be moved about and between the radially retracted condition and the radially expanded condition by axially moving (retracting) a guide member that is secured to the splines such that the splines are deformed to the radially expanded condition. Optionally, in exemplary aspects and as shown in FIG. 12B, the plurality of electrodes 10 can comprise a central reference electrode 12 positioned within a central area 56 defined by the plurality of splines 52 of the catheter 50. In further exemplary aspects, when the plurality of splines are in the radially expanded condition, the central reference electrode can be equidistant from the at least three non-collinear electrodes. Optionally, as shown in FIGS. 12A-12B, it is contemplated that each spline 52 can support at least one electrode (optionally, a plurality of electrodes).

In exemplary aspects, the processor 30 can be provided as part of a computer, a remote handheld device, a tablet, a smartphone, and the like. In these aspects, it is contemplated that the processor 30 can be communicatively coupled (e.g., through a wired or wireless connection) to a memory and/or a remote or Cloud-based network that provides the processor with access to stored electrical stimulation protocols, patient data, and other information necessary to perform the methods disclosed herein. Thus, it is contemplated that the processor can be configured to run programs that are stored on a memory or network that is in communication with the processor. Such communication can be through any conventional means, including both wireless and wired connections. In exemplary aspects, the processor 30 can comprise a plurality of processing units or modules that are configured to perform various functions, such as, for example and without limitation, processing of the signals received from the electrodes, activation of the pulse generator, and the like. Although disclosed herein as a single processor 30, it is contemplated that the disclosed system can include more than one processor that cooperate to perform the functions of the processor 30 disclosed herein. In further exemplary aspects, it is contemplated that the processor 30 can be positioned in communication with a user interface, such as a keyboard, a touchscreen display, a computer mouse, a joystick, a remote control, a handheld device, a tablet, and the like, that is configured to receive inputs from a user related to the pacing or recording of electrical activity within cardiac tissue as further disclosed herein. For example, in exemplary aspects, it is contemplated that the user interface can comprise knobs that can be selectively positioned to modify selected pacing wave parameters.

In exemplary aspects, and as further disclosed herein, the electrodes 10, the pulse generator 20, or the processor 30 can be provided as components of an electrophysiological recording system as is known in the art and further disclosed herein, such as, for example and without limitation, the CARTO® system (Biosense Webster), the EnSite™ system (St. Jude Medical), or the Rhythmia™ system (Boston Scientific). Optionally, in some aspects, the electrodes 10, the pulse generator 20, and the processor 30 can be provided as components of such an electrophysiological recording system.

In use, it is contemplated that the plurality of electrodes can comprise bi-polar electrodes as are known in the art. In exemplary aspects, and as further disclosed herein, it is contemplated that it can be advantageous to position the bi-polar electrodes in substantially perpendicular orientations (e.g., perpendicular or within about 15 degrees of perpendicular) relative to the myocardial tissue. In these aspects, it is contemplated that such orientations can produced insensitivity to the direction of the activation impulse and enhanced sensitivity and specificity for the detection of abnormal myocardium.

As further disclosed herein, it is contemplated that the disclosed systems and methods can provided improved accuracy of voltage mapping through the use of perpendicular or substantially perpendicular orientations of bi-polar electrodes. Such improvements can be interpreted in terms of EGM referencing. With perpendicular or substantially perpendicular electrode orientations as disclosed herein, it is contemplated that the disclosed systems and methods can effectively produce near-field EGM measurements, with a distal (exploratory) electrode contacting tissue and a reference electrode in the blood pool no closer to any cardiac tissue than the tissue being probed by the exploratory electrode. Beneficial properties of such near-field EGM measurements include features typically associated with bipolar EGMs, such as common mode rejection of far-field signals, and with unipolar EGMs, such as consistent EGM morphology and insensitivity to the direction of wavefront propagation. It is contemplated that such near-field EGM measurements can be used effectively in clinical electrophysiology applications, in particular those relating to substrate mapping. In exemplary aspects, it is contemplated that the basket electrode catheter 50 described herein with reference to FIGS. 12A-12B can be used to inherently acquire near-field EGMs wherever an exploratory electrode comes in contact with myocardial tissue. In particular, the placement of the reference electrode at the center of the basket array can allow all EGMs to be acquired with near-field EGM referencing. The splines of the basket thereby form a protective barrier that prevents the central reference electrode from coming into contact with the cardiac tissue. Additionally, when the reference electrode is equidistant from the exploratory electrodes, the reference can be consistent for all channels of the recording assembly.

As will be appreciated by one skilled in the art, the disclosed methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

In exemplary aspects, computer program instructions may be loaded onto a general purpose computer, special purpose computer, or other programmable data processing apparatus to produce a machine, such that the instructions which execute on the computer or other programmable data processing apparatus create a means for implementing various functions as further disclosed herein.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the various functions disclosed herein.

In an exemplary aspect, and as further disclosed herein, the methods and systems can be implemented using a computer as described below. Similarly, the methods and systems disclosed can utilize one or more computers to perform one or more functions in one or more locations. The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer, which can comprise, but is not limited to, one or more processors 30, a system memory, and a system bus that couples various system components including the one or more processors to the system memory. The system can utilize parallel computing.

The system bus represents one or more of several possible types of bus structures, including a memory bus or memory controller, a peripheral bus, an accelerated graphics port, or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus, and all buses specified in this description can also be implemented over a wired or wireless network connection and each of the subsystems, including the one or more processors 30, a mass storage device, an operating system, impression creation software, impression creation data, a network adapter, the system memory, an Input/Output Interface, a display adapter, a display device, and a human machine interface, can be contained within one or more remote computing devices at physically separate locations, connected through buses of this form, in effect implementing a fully distributed system.

The computer typically comprises a variety of computer readable media. Exemplary readable media can be any available media that is accessible by the computer and comprises, for example and not meant to be limiting, both volatile and non-volatile media, removable and non-removable media. The system memory comprises computer readable media in the form of volatile memory, such as random access memory (RAM), and/or non-volatile memory, such as read only memory (ROM). The system memory typically contains data such as the impression creation data and/or program modules such as the operating system and the impression creation software that are immediately accessible to and/or are presently operated on by the one or more processors 30.

In another aspect, the computer can also comprise other removable/non-removable, volatile/non-volatile computer storage media. By way of example, a mass storage device can provide non-volatile storage of computer code, computer readable instructions, data structures, program modules, and other data for the computer. For example and not meant to be limiting, the mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device, including by way of example, the operating system and the impression creation software. Each of the operating system and the impression creation software (or some combination thereof) can comprise elements of the programming and the impression creation software. The impression creation data can also be stored on the mass storage device. The impression creation data can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the one or more processors 30 via the human machine interface that is coupled to the system bus, but can be connected by other interface and bus structures, such as a parallel port, game port, an IEEE 1394 Port (also known as a Firewire port), a serial port, or a universal serial bus (USB).

In yet another aspect, the display device can also be connected to the system bus via an interface, such as the display adapter. It is contemplated that the computer can have more than one display adapter and the computer can have more than one display device. For example, the display device can be a monitor, an LCD (Liquid Crystal Display), or a projector. In addition to the display device, other output peripheral devices can comprise components such as speakers (not shown) and a printer (not shown) which can be connected to the computer via the Input/Output Interface. Any step and/or result of the methods can be output in any form to an output device. Such output can be any form of visual representation, including, but not limited to, textual, graphical, animation, audio, tactile, and the like. The display device and computer can be part of one device, or separate devices.

The computer can operate in a networked environment using logical connections to one or more remote computing devices. By way of example, a remote computing device can be a personal computer, portable computer, smartphone, a server, a router, a network computer, a peer device or other common network node, and so on. Logical connections between the computer and a remote computing device can be made via a network, such as a local area network (LAN) and/or a general wide area network (WAN). Such network connections can be through the network adapter. The network adapter can be implemented in both wired and wireless environments. Such networking environments are conventional and commonplace in dwellings, offices, enterprise-wide computer networks, intranets, and the Internet.

In operation, the application programs and other executable program components such as the operating system can reside at various times in different storage components of the computing device and be executed by the one or more processors 30 of the computer. An implementation of the impression creation software can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

### Exemplary Applications

In still a further aspect, the disclosed methods can characterize the underlying tissue under conditions that can contribute to arrhythmogensis. For example, a programmed stimulation pacing strategy can unmask the conductive properties of the myocardium that exist during premature stimulation or extrasystole. During programmed stimulation, the heart can be excited with a train of paced beats that can condition the electrophysiology of the heart to a given rate. It is contemplated that multiple consecutive beats with a fixed interval can be provided to allow the tissue to reach an electrophysiological steady state before application of a paced beat at a shorter coupling interval. The run of consecutive beats can also allow confirmation that the pacing stimulus is appropriately capturing or exciting the myocardium before delivery of the early stimulus. Once the heart is sufficiently conditioned, usually after eight successive paced beats, a final paced beat can be delivered at a shorter coupling interval. This early beat mimics the occurrence of premature beats in the heart that are frequently the trigger for cardiac arrhythmias. Thus, the disclosed systems and methods can measure the conductive properties of the heart as they would appear under the stress of premature excitation.

In another aspect, it is contemplated that longitudinal and transverse fiber orientations of the tissue that are enclosed in the catheter (e.g., loop catheter) can be reconstructed based on CVs derived from paced excitations. The feasibility of fiber orientation reconstruction using a singular value decomposition of the CV vectors computed from the pacing protocol is demonstrated herein. First, electrograms can be acquired from all non-pacing electrodes, as each adjacent pair of electrodes around the loop catheter can be sequentially paced. The electrograms can be assessed for activation times based on the maximum negative deflection of the signal that occurs as the action potential passes under the recording electrode. Using the known locations of both the pacing and recording electrodes, as tracked by modern electrophysiology systems, a CV vector can be computed as a function of the distance between the pacing and recoding electrodes divided by the time from stimulus onset at the pacing electrode to the activation time at the recording electrode. This quantity represents the magnitude of the CV vector. The orientation of the CV vector can be the vector from the pacing electrode location to the recording electrode location. This CV vector and all subsequently computed velocity vectors can then be mapped to a point at the center of the loop array. The anisotropy of the tissue can then be characterized by computing the singular value decomposition of the CV vectors. The described function can produce pseudo-eigenvalues and eigenvectors, the first of which can point in the direction of maximum variation and can be taken as the longitudinal direction of conduction. The second eigenvalue and vector are guaranteed to be orthogonal to the first and correspond to the transverse direction of conduction.

Multiple disease processes can lead to the formation of cardiac arrhythmias. However, regardless of the source, arrhythmogenesis nearly always can be distilled to two primary factors, a trigger and a substrate. Triggers most often come in the form of early depolarizations that excite the cardiac tissue before it is prepared to uniformly accommodate an action potential. However, premature excitation can occur frequently even in healthy hearts, and usually to not induce arrhythmias. In order for triggers to produce arrhythmia, they must interact with a proarrhythmic substrate that can support the arrhythmia. Structural aspects of the cardiac tissue such as scarring or fibrosis can act as a substrate for arrhythmogenesis. Additionally, electrophysiological features of the tissue, such as repolarization heterogeneity, can also serve as the substrate. The clinical utility of the tissue interrogation strategy described herein lies, at least in part, in its ability to characterize the response of the tissue substrate to premature excitations. The measure of absolute conduction velocities as they relate to the fiber structure of the heart can identify regions of myocardium that will exhibit proarrhythmic behavior when exposed to triggers, e.g., conduction block. Identification of these regions can both facilitate mechanistic explication of arrhythmogenic processes on a patient specific level and also aid in targeting of preventative therapies.

### EXPERIMENTAL EXAMPLES

The following examples are put forth so as to provide those of ordinary skill in the art with a complete disclosure and description of how the devices, systems, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, lengths etc.), but some errors and deviations should be accounted for.

### Example 1

The onset and entrenchment of atrial fibrillation (AF) is strongly associated with remodeling of the atrial myocardial substrate. As a result, as remodeling progresses, ablation strategies based solely on compartmentalization lose efficacy. Consequently, great clinical interest has focused on substrate mapping strategies that can identify proarrhythmic tissues for targeted therapy. Such strategies employ intracardiac electrogram (EGM) parameters, such as voltage amplitude and presence and degree of fractionation, as markers of tissue with abnormal conductive properties. However, these traditional mapping strategies for AF, whether recorded in sinus rhythm or AF, have neglected the effect of variable activation patterns on EGM parameters of interest. A key feature of an ectopically triggered beat in the heart is that the resulting activation does not follow the same conduction patterns as a normal sinus beat. Thus, sinus conduction may mask the remodeled and pro-arrhythmic substrates that can only be exposed by extrasystole. Thus, Example 1 describes an experimental procedure for mapping electrophysiological substrate features using varied activation patterns.

In this experiment, the feasibility of mapping electrophysiological substrate features using varied activation patterns was demonstrated by pacing and recording within the same region of the heart using a clinical loop catheter. To demonstrate the feasibility of substrate mapping with controlled activation patterns, CV was selected as the initial parameter of interest. Conduction velocity is an important factor in the initiation of re-entry and is affected by substrate remodeling (heterogeneity and slowing). As disclosed herein, this experimental procedure explored factors associated with measurement of CV and evaluated the information such measurements provide about myocardial substrate.

Methods

The experimental procedure incorporated computation modeling and direct recording of electrograms in large mammals. In both cases, a pacing protocol involving stimulation from bipolar electrodes on a 10 pole loop catheter was performed to interrogate the conduction properties of the tissue. Specifically, a depolarization wave was activated by pacing from each pair of adjacent electrodes on the loop catheter, i.e., 1-2, 2-3, . . . , 9-10, to stimulate the region contained by the loop with nine different activation patterns. EGMs from the non-pacing electrodes were acquired for each activation pattern and from them, activation times were determined from unipolar EGMs by computing the maximum negative slope, or from bipolar EGMs by a nonlinear energy operator. CV was calculated as the distance, from the pacing site to the recording electrode, over the time to activation. CVs and directions from the respective studies were visualized using SCIRun (NIH/NIGMS Center for Integrative Biomedical Computing, Salt Lake City, Utah).

Simulation

A three-dimensional bi-domain simulation of the spread of activation from pacing sites on slab models of atrial tissue was implemented with the Cardiac Arrhythmia Research Package (CARP) software. The models were 26.0×26.0×3.0 mm slabs composed of isotropic hexahedral elements with 0.1 mm edge lengths immersed in 1.0 mm of bath surrounding all slab surfaces. For each activation pattern, a complete activation of the slab was computed using the Courtemanche-Ramirez-Nattel cell model of atrial myocyte kinetics. Pseudo-EGMs were generated at each electrode (1 kHz sampling frequency) for the entire 50 ms of simulation. Model conductivities were varied to produce both isotropic and anisotropic models, as shown in FIG. 1.

Characterization

The relationship between conduction velocity and the distance of the recording electrode from the pacing site was plotted and fit by linear regression. The slope of this regression was then used to normalize all CVs as though they had been recorded 20 mm from the pacing site.

CVs and their respective directions of conduction were compared to the fiber orientation of the computational models. Specifically, the angle between the fiber axis and directions of minimum and maximum conduction velocity were qualitatively assessed for correlation.

In Vivo Experimentation

The feasibility of pacing and recording from a single loop catheter for assessment of conduction velocities was tested during in vivo experimentation on a single canine. A 10 pole trackable circular mapping catheter (LASSO®NAV manufactured by Biosense Webster, Inc.) was introduced into the right atrium via percutaneous access of the femoral vein. The electrode array was placed in the high right atrium on the lateral wall and positioned such that clear EGMs were visible on all channels. Pacing was achieved with 600 ms cycle length, 10 mA, and 2 ms pulse width stimulation. Myocardial capture was confirmed, and then location and electrogram recordings were acquired with a CARTO® 3 electrophysiological recording system. It is contemplated that the reconstruction of myocardial conductance properties can be accomplished with nearly any known electrophysiology mapping system that can track electrode locations and record electrograms, e.g., the CARTO® system from Biosense Webster, the EnSite system by St. Jude Medical, or the Rhythmia system from Boston Scientific. Additionally, there is no specific requirement that the recording array be loop shaped or have a specific number of electrodes. The minimum requirement necessary to attempt this mapping strategy is that the recording array must contain three or more non-collinear electrodes that can simultaneously be in contact with the myocardium. It is contemplated that an increase in the number of electrodes can improve the resolution achievable with the systems and methods disclosed herein.

Results

Simulation

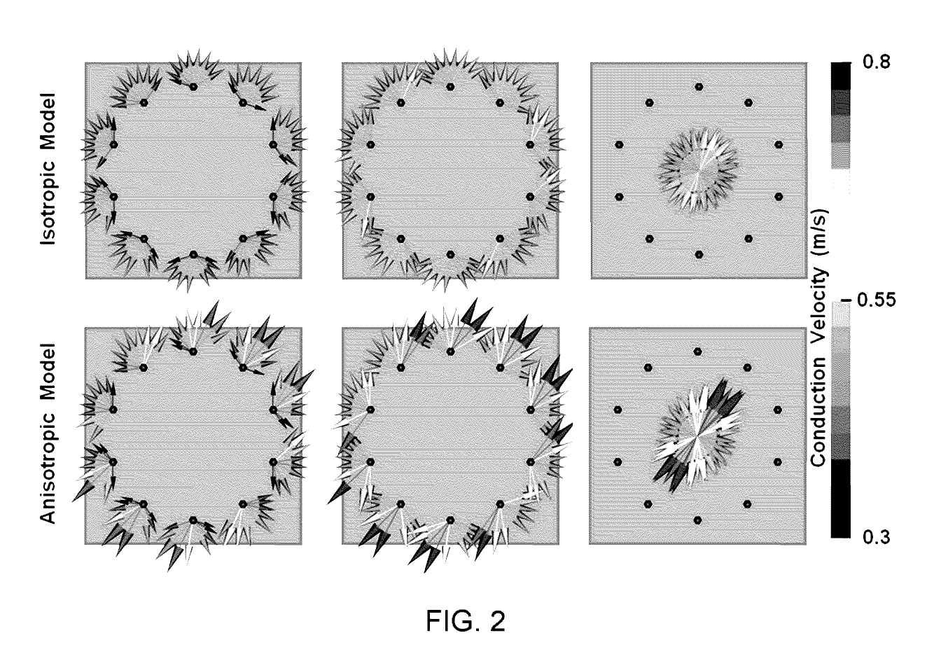

Activation times were found for all loop catheter electrodes in the computational model. As shown in FIG. 2 (left column), CV and the direction of conduction were plotted as color-mapped vectors over the model, with both the color and magnitude corresponding to the CV. In both the isotropic and anisotropic models, CVs that were measured from electrodes in proximity to the pacing site showed slower CVs than were observed at distant recording electrodes.

Characterization of Conduction

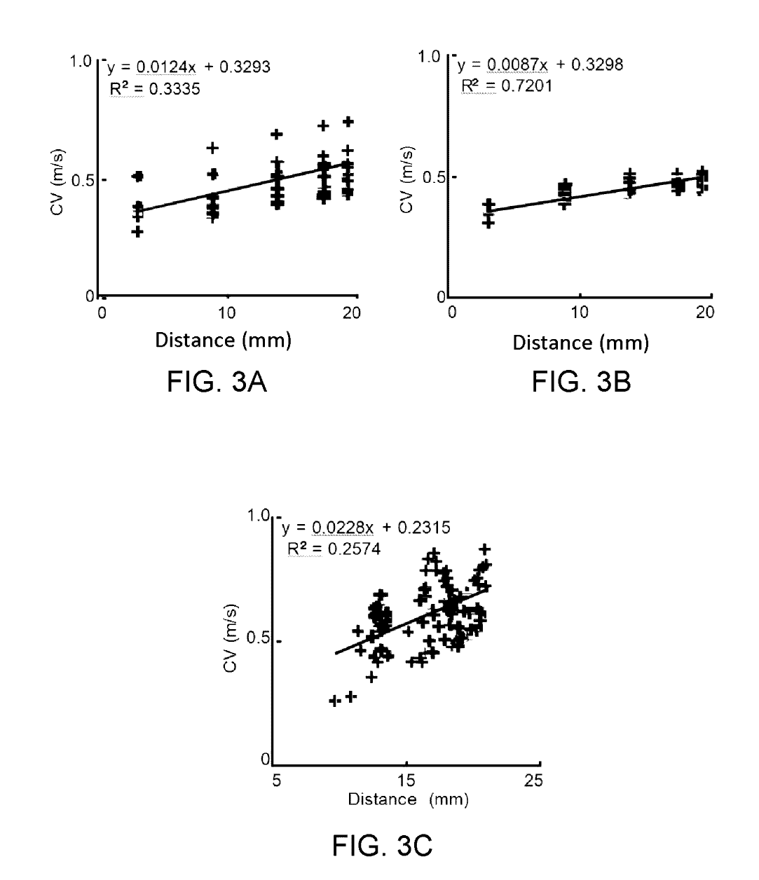

FIGS. 3A-3C show a linear regression of conduction velocity as a function of conduction distance. As shown in the middle column of FIG. 2, the CVs can be normalized to a fixed distance of 20 mm by the slopes of the respective regressions. In the isotropic model (FIG. 2, top row) the pre-normalization vectors (FIG. 2, left column) can demonstrate anisotropy of CVs inconsistent with the spread of activation in the model. The normalized vectors (FIG. 2, middle column), in contrast, can indicate a uniform spread of activation consistent with the model conductivities. Anisotropy is apparent before normalization in the CVs of the anisotropic model; however, the CVs from electrode to electrode can be inconsistent, even when oriented in the same direction, due to variable proximity of the recording electrodes to the paced sites. The normalization can improve the consistency of CVs among vectors pointing in the same direction (visible in the center and right column of the second row of FIG. 2). In the anisotropic model, the direction of highest CV lay along the fiber orientation (see FIG. 1). As a compact representation of the conduction properties, the normalized vectors from each recording site were projected onto a point at the center of the loop catheter (FIG. 2, right column).

In Vivo Experimentation

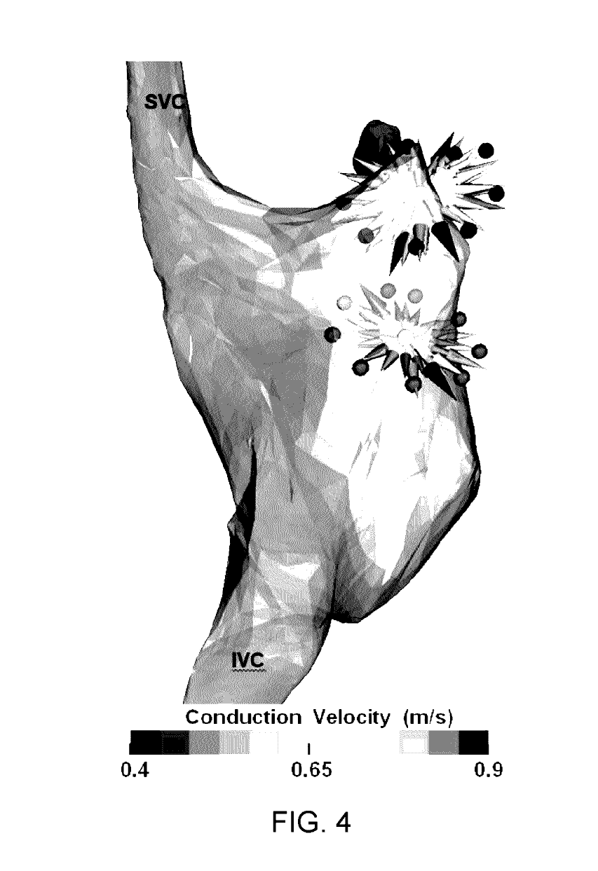

The pacing protocol to stimulate myocardial tissue with multiple activation patterns was successfully carried out at 2 locations in the canine model. Activation times were acquired on a median of 5 (min=3, max=6) bipolar channels for each pacing site. The number of interpretable EGMs was limited due to pacing artifact that saturated channels near the pacing site or by channels with no detectable activation signals. Similar to the simulation results, CV also increased with distance of the recording electrode from the pacing site. FIG. 4 shows the result of normalizing and plotting CV vectors from measurements from two locations on the RA of the animal.

Discussion

The results of the experimental procedure disclosed herein show that interrogation of regions of myocardium with multiple activation patterns can elucidate basic conduction properties of that tissue, e.g., CV and anisotropy, in a way that corrects for possible artifacts from the location of the pacing cite. The results of such evaluations of the myocardial substrate can be useful to establish patient specific ablation strategies for arrhythmias like AF.

The rate of conduction from a focal pacing site is not constant even in the immediate area around the pacing site, and increases as the depolarization wave propagates outward. Consequently, electrodes in proximity to the pacing site will observe relatively slower conduction than distant electrodes. The normalization by distance disclosed herein allows for inter-electrode comparison of CVs and can improve the characterization of local conduction properties. The maximum conduction distance for such models was about 20 mm, and the acceleration of conduction remained relatively constant over these distances.

Previous studies have reported using loop catheters to record CV from planar wavefronts during sinus conduction; however, those results have been indicative of conduction in the longitudinal direction only, i.e., parallel to the predominant fiber orientation. More generally, wavefronts initiated by local pacing have longitudinal and transverse aspects that permit characterization of the anisotropy of conduction. The present disclosure demonstrates how both longitudinal and transverse CVs may be recovered with this protocol.

The primary tenet of this controlled activation pattern mapping protocol is that sinus conduction is inherently stable and not prone to arrhythmogenesis. Thus, electroanatomical substrate mapping of tissue properties during sinus rhythm can fail to identify pro-arrhythmic substrates that are unmasked by ectopy and non-sinus conduction. Robust characterization of the atrial conduction substrate, as disclosed herein, can facilitate better understanding of the mechanisms behind AF initiation and provide better targets for therapeutic interventions.

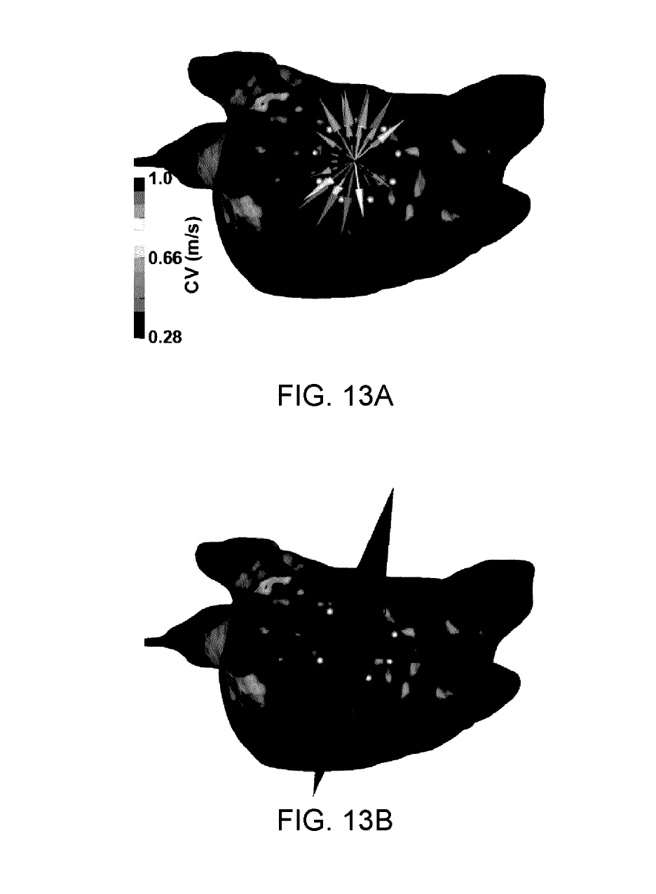

Example 1 demonstrates that the normalized vectors can be projected to the center of the loop array of a loop catheter. The collection of projected vectors contains a great deal of information about the local conduction properties, but it is difficult to interpret relevant information from this representation, i.e. longitudinal and transverse CV or conduction anisotropy. Disclosed herein is a method for simplifying this information is a dimensionality reduction-based approach known as singular value decomposition (SVD). Very similar to other decomposition strategies, e.g., eigenvalue, or Fourier decomposition, an SVD of the vector data can identify the principal axes of variation in the data. This approach can identify, as the first principal axis, the direction in which action potential impulses conduct the fastest, i.e., the direction of longitudinal CV (FIGS. 13A and 13B). The next component or axis can be perpendicular to the first axis and can provide an approximation of the transverse CV. The disclosed method for evaluating cardiac conduction properties can be repeated throughout the heart and is feasible with any catheter technology known in the art.

### Example 2

As described in Example 1, CV and the anisotrophy of conduction are important indicators of proarrhythmic potential. Example 1 describes a method of CV measurement that involves activation times for every electrode measuring impulse propagation. Also disclosed herein, however, are methods and systems for measuring CV and conduction anisotrophy, without the requirement of activation time assignment, by assessing the mean Laplacian electrogram as computed over a clinically feasible electrode array.

Slowing of cardiac CV and associated changes in the anisotropy of conduction are important factors of the arrhythmogenic substrate and when evaluating pro-arrhythmia risk. However, there are currently no systems or methods available to one of skill in the art to estimate cardiac conduction and anisotrophy in a fast and reliable manner. Described herein are methods in which a single electrical signal, the mean Laplacian electrogram (MLE), constructed from a number of simultaneously recorded unipolar electrograms, can be used to quantify both velocity and anisotropy of conduction. Transverse and longitudinal CVs can be quantified directly from MLE landmarks that can be identified manually or by a computer program. The approach works directly on the MLE and thereby circumvents the need to assign activation times to individual uni- or bipolar electrograms. Further, the described methods do not require visualization or knowledge of the spatial propagation of the wavefront. The disclosed methods can be used in clinical, cardiac electrophysiology laboratories during substrate mapping and ablation procedures to assess local conduction velocities quickly and reliably.

In this experimental procedure, data was obtained by electrical mapping of isolated guinea pig hearts using a multi-electrode array. Computer simulations were performed in a three-dimensional bi-domain model incorporating tissue anisotropy. For both experimental and simulated data, CV and anisotropy were calculated and compared to distinct features of the MLE which, as described herein, is a spatial average of Laplacian electrograms.

The QRS region of the MLE showed two distinct peaks. In animal experiments, the temporal locations of the peaks were sensitive to pharmacological manipulation of conduction. The first and second peak correlated well with longitudinal (r=−0.52) and transverse (r=−0.82) CV, respectively. The simulations demonstrated that the MLE was dependent on the angle of rotation of the electrode array relative to fiber orientation. Mathematical analysis resulted in an optimized array design without rotational dependence. The simulations were further used to link the morphology of the optimized MLE to the spatial development of the wavefront, resulting in highly accurate descriptors of longitudinal and transverse CV, as well as anisotropy.

### Example 3

The conductive properties of cardiac tissue are an important part of the arrhythmogenic substrate. Various diseases, e.g. ischemia, infarction, and fibrosis, alter the conductive properties of the myocardium, creating favorable conditions for ectopy and reentry. Specifically, cardiomyopathies that cause a decrease in CV favor arrhythmogenesis by decreasing the critical wavelength necessary to foster reentry. However, the CV of an excitation wave moving through myocardial tissue can depend on the alignment of the wave direction and the underlying orientation of the muscle fibers. Conduction along the fiber direction (longitudinal) will progress much faster than conduction across the fibers (transverse). Although this anisotropy is a normal feature of cardiac conduction, abnormal anisotropy can result in conduction block and the generation of reentrant arrhythmias.

Changes to the anisotropy of CV have been observed with aging and in conjunction with cardiomyopathies that promote microfibrosis and remodeling of gap junction expression and localization. In the experimental laboratory, multidetector arrays, e.g, plaque electrodes or optical mapping systems, can facilitate the creation of activation maps at high temporal and spatial resolution that track the spread of activation through the myocardium. Such approaches allow calculation of longitudinal CV (CVL), transverse CV (CVT), and thus anisotropy. However, these techniques are not easily adaptable for clinical use as they do not provide a means for fast and reliable estimation of CV and anisotropy in clinical electrophysiology (EP) studies. Thus, in one embodiment, disclosed herein are methods of robust clinical characterization of CV and anisotropy during minimally invasive EP studies for characterizing proarrhythmic substrates in patients.

Laplacian electrograms (LEs) are typically computed from a grid of unipolar electrodes (UEs) in an approximation of the second order spatial gradient of the surface potential, which results in a local estimate of current sources or sinks. This approximation can allow for robust detection of cardiac activation times. Thus, a signal based on spatial averaging of LEs can provide information about the local spread of activation and anisotropy of CV.

Described in Example 3 is a method for combining information from a grid of UEs into a single signal, the mean Laplacian electrogram (MLE). It was shown that distinct features of the QRS region of the MLE signal correlate directly with local cardiac conduction velocities in the underlying tissue. Computer simulations were also performed in order to elucidate the electrophysiological underpinnings of the signal, as described herein.

Methods

Experimental Procedures

Langendorff perfused guinea pig hearts were electrically mapped by recording UEs from the anterior epicardial surface. The recording electrodes were ordered in a regular 8 by 8 grid with 2 mm interelectrode spacing. One of the four central electrodes was used to pace the heart at a cycle length of 300 ms. The signals were high- and low-pass filtered at 0.03 and 500 Hz, respectively, and digitally sampled at 4 kHz. Hearts were perfused with a solution consisting of 1.25 mM CaCl2, 140 mM NaCl, 4.5 mM KCl, 5.5 mM dextrose, 0.7 mM MgCl2, and 10 mM HEPES (pH 7.4). Conduction was slowed by adding either 1 μM flecainide (USP, Rockville, Md.) or 30-50 μM carbenoxolone (Sigma-Aldrich, St. Louis, Mo.) to the perfusate.

Simulation Procedures

All simulations were performed using a three-dimensional bi-domain slab model of electrically anisotropic myocardial tissue created using the SCIRun problem solving environment and implemented using Cardiac Arrhythmia Research Package (CARP) software. The dimensions of the slab were 26×26×3 mm and it was composed of isotropic hexahedral elements with 0.1 mm edge length immersed in 1.0 mm of bath solution surrounding all slab surfaces. The excitable properties of the model were based on the cellular model of Courtemanche et al (1998).

Conduction velocity and anisotropy were varied by scaling the longitudinal and transverse conductivities independently. Extracellular potentials corresponding to UEs were computed at different locations and evaluated at a sampling rate of 1 kHz.

Data Analysis

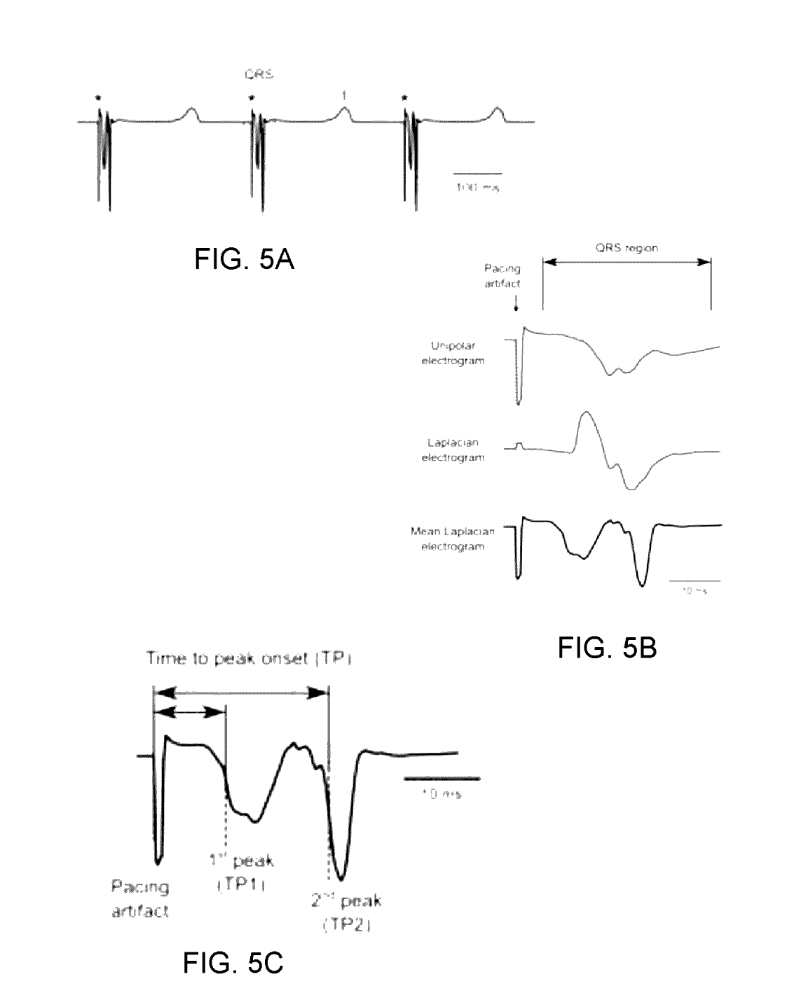

CVs were calculated based on activation time maps, as described herein. Local Laplacian potentials were calculated according to Janse et al. (1980), with the exception of a unit conversion. Local Laplacian electrograms (LE) were calculated for electrodes for which there were at least 7 surrounding electrodes that could be included in the calculation. This restriction excluded the outermost electrodes in the grid and the pacing electrode, resulting in a total of 35 LEs. The 35 LEs were then averaged to obtain a single signal, which is referred to herein as the mean Laplacian electrogram (MLE). The temporal locations of the two separate peaks in the QRS region of the experimentally measured MLE were parameterized by the time to peak onset (TP) of each peak. By analogy to activation time assignment in a UE, the TP was defined as the time from the pacing artifact to the maximum negative down stroke of the relevant peak, as shown in FIG. 5C.

Statistical Procedures

All statistical calculations were performed using R, specifically the functions ‘stats’ (v3.0.2), ‘boot’ (v1.3-9), ‘smatr’ (v3.4-3) and ‘simpleboot’ (v1.1-3). The correlation between the two measures of conduction (CV vs TP) was quantified using the sample Pearson correlation coefficient (r). Bootstrap distributions of the r-values were generated based on 5000 bootstrap replicates, and 95% confidence intervals (CI, bias-corrected and accelerated) were estimated. A correlation was deemed significant if the CI did not include 0 and was labeled according to the r-value as very strong (r>0.8), strong (0.8>r>0.6), moderate (0.6>r>0.4), or weak (r<0.4). For experimental data, differences in means were compared using Welch's t-test. Unless otherwise specified, experimental data are summarized by the mean and standard deviation (SD). For the simulation data, CV estimates obtained with the standard vector based approach were compared directly with the MLE approach by means of a type II linear regression (major axis) analysis, and 95% CIs were estimated for the fitted parameters (slope and intercept). If the CI for the slope included 1, it was taken to indicate no significant bias between variables.

Results

Quantification of Conductivity from the MLE

A method for obtaining a reliable estimate of conduction velocity and anisotropy in cardiac tissue that can be implemented in the clinical EP laboratory was identified. To this end, the properties of the mean MLE, which is the average of multiple Les, was explored. FIG. 5A shows an MLE recorded from a guinea pig heart during epicardial pacing from the center of the recording array. A total of three beats are shown. Further, FIG. 5A shows that the MLE displayed recognizable deflections similar to a QRS complex and a T-wave. FIG. 5B shows the QRS region of a representative UE, the LE for the same electrode, and the resulting MLE for comparison. The MLE displayed two very distinct downward deflections referred to as peaks. To analyze the origin of the peaks in the spatially averaged signal, the temporal locations of the peaks were quantified by the time to peak onset (TP) defined as the time from the pacing artifact to the maximum negative downstroke of the peaks. Thus, as shown in FIG. 5C, TP1 and TP2 refer to the temporal locations of the first and second peak, respectively.

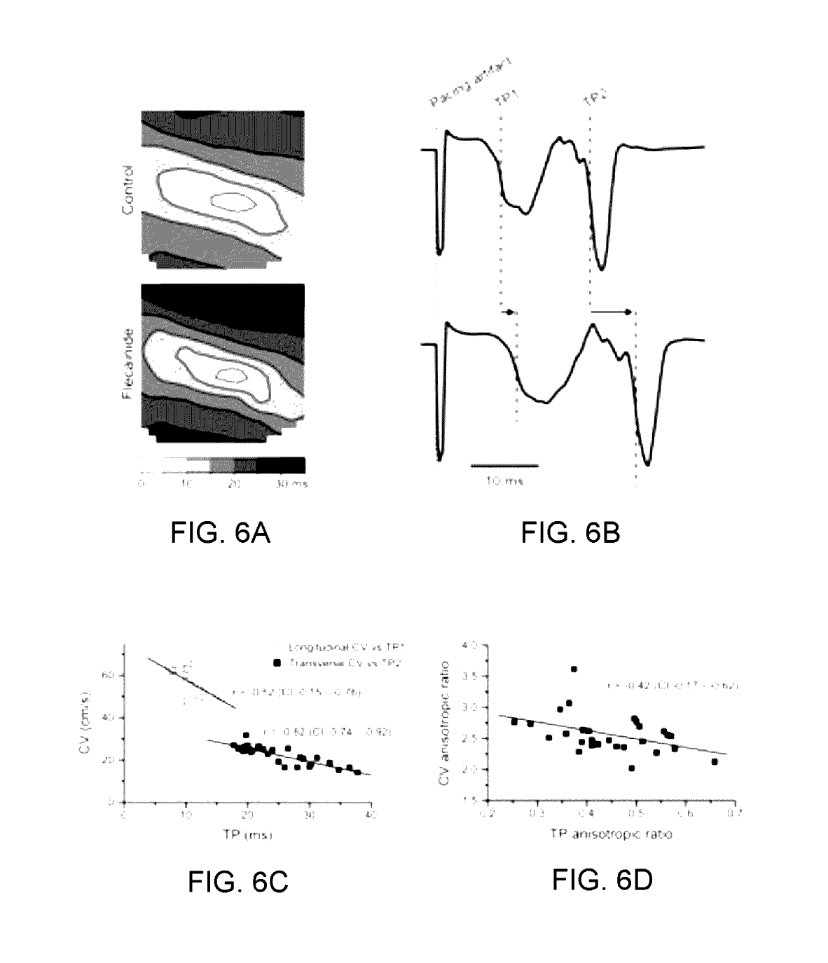

The LE represented local current singularities (positive deflections are current sources and negative deflections are current sinks) in the tissue underneath the recording electrode, and the peaks in the QRS region of the MLE represented the major current sinks in the tissue under the recording array. Further, the peaks were representative of the principal components of current flow in the tissue and thus their temporal location was sensitive to changes in CV. In this experiment, conduction was pharmacologically slowed using flecainide, a sodium channel blocker, or carbenoxolone, a gap junction uncoupler, in guinea pig hearts. FIG. 6A shows activation time isochrone maps from a representative heart recorded under control (top) and flecainide (bottom) conditions. Conduction slowing in the presence of flecainide was evident from the closer spacing of the isochrones compared to the control. The corresponding MLEs are shown in FIG. 6B, with the temporal locations of both peaks shifted to the right. Next, it was shown that the temporal location of the peaks correlated with transverse and longitudinal components of conduction velocity. In FIG. 6C, CVL is plotted against TP1 (open squares) and CVT is plotted against TP2 (filled squares) for control (n=15), flecainide (n=5), and carbenoxolone (n=8) conditions. For the correlation analysis to be valid, independent data values were used. Thus, the analysis presented in FIG. 6C includes only unpaired data. For the same reason, longitudinal and transverse data were analyzed separately. For CVL versus TP1, the correlation coefficient was moderate (r=−0.52 (CI −0.15-−0.76)), while correlation between CVT and TP2 was strong (r=−0.82 (CI −0.74-−0.92)). The relationship between CV anisotropy (ratio of CVL to CVT) and the ratio of TP1 to TP2 is shown in FIG. 6D. Although the correlation was only moderate (r=−0.42 (CI −0.17-−0.62)), which is likely due to the variation in TP1 (see FIG. 6C), the peaks in the QRS region of the MLE capture the conductive properties of the tissue.

To evaluate whether the MLE derived measurements can also be used to detect conduction slowing directly, a comparison of experiments was performed in which paired data (n=13) was available for both control and pharmacologically slowed conduction. First, evaluating conduction velocity in the standard way, significant reductions of 21±6% (p<0.001) and 30±10% (p<0.001) were detected for CVL and CVT, respectively. These numbers compared well against changes in TP1 and TP2 of 20±18% (p=0.003) and 40±20% p<0.001), respectively, demonstrating that the MLE can be used to detect conduction slowing.

Reproduction of the MLE by Computer Simulations

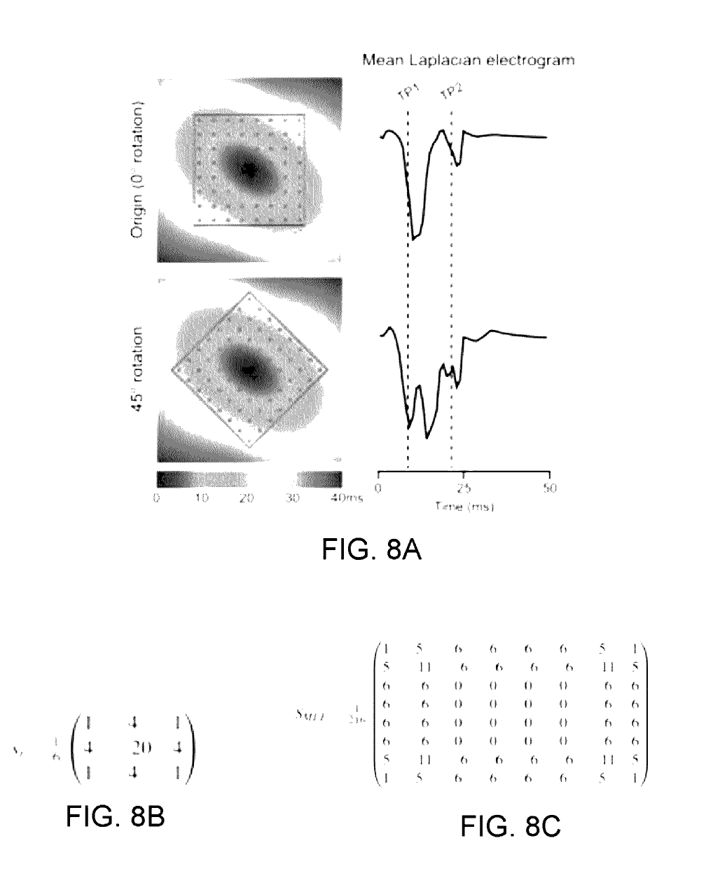

To better understand the electrophysiological origin and meaning of the MLE, computer simulations of a slab model of cardiac tissue were performed, and the signals were reconstructed from a 8×8 electrode grid similar to the one used in the ex vivo experiments. FIG. 8A (top panel) shows the activation time map from a representative simulation and corresponding MLE with the electrode array rotated in two different angles with respect to fiber orientation (denoted 0 and 45 degrees, top and bottom panels, respectively). For the 0 degree orientation, which is close to the orientation of the array in the ex vivo measurements, the reconstructed MLE similarly displayed two distinct peaks. However, rotation of the electrode array resulted in a more complex morphology of the MLE. The mathematics behind the calculation of the MLE was also examined. Since each individual LE was calculated using a 3×3 coefficient stencil (FIG. 8B), the LE can effectively be considered a weighted linear combination of the nine electrodes included in the stencil. Consequently, as the MLE is a simple spatial average of the Les, it too can be obtained directly from a linear combination of electrodes. Thus, instead of calculating the MLE as the average of 35 LEs, the corresponding mathematical expression can be rearranged and simplified substantially. The resulting expression can be organized in a matrix representation including all electrodes on the grid. The equivalent 8×8 coefficient stencil is shown in FIG. 8C. The stencil showed that the central electrodes do not contribute to the MLE (coefficients of zero) and that there is a markedly heterogeneous contribution among the electrodes with non-zero coefficients. This unequal contribution of particularly the corner electrodes explains the rotational dependence of the MLE based on the 8×8 electrode array.

Development of a Clinically Adaptable Electrode Configuration

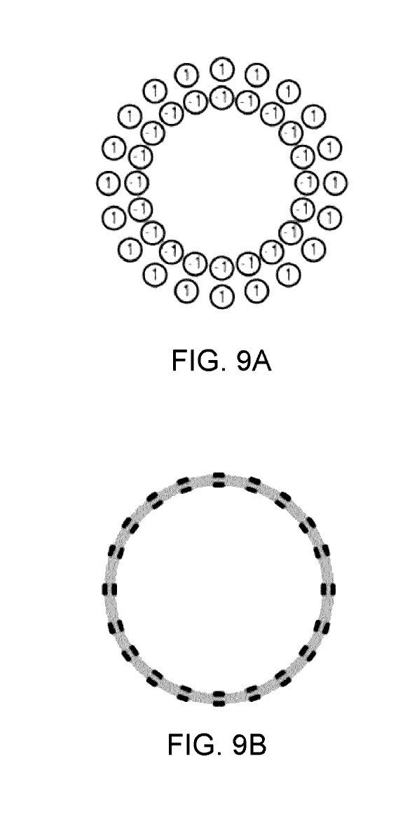

For clinically practical purposes, it is contemplated that a circular configuration of the electrodes can be more easily implemented in a catheter-based solution than a rectilinear grid. A potential solution can be obtained by considering only the electrodes with non-zero coefficients in the stencil shown in FIG. 8C. These non-zero electrodes were identical to two squares with electrodes along the sides: an outer square with electrode coefficients of positive signs and an inner square with electrode coefficients of negative signs. Transforming the shape of the squares into two concentric circles resulted in a design similar to the one shown in FIGS. 9A and 9B. FIG. 9A shows the localization and coefficients of two concentric rings, referred to as a dual ring array, with 20 electrodes each. The resulting MLE can be calculated as the sum of the potentials recorded on the outer ring minus the sum of the potentials recorded on the inner ring, according to the following formula:

$$MLE = ( {\sum }_{o = 1}^{n} {φ}_{o} - {\sum }_{i = 1}^{n} {φ}_{i} ) / n$$

Such an electrode configuration can be implemented on a fixed diameter circular recording catheter as shown in FIG. 9B.

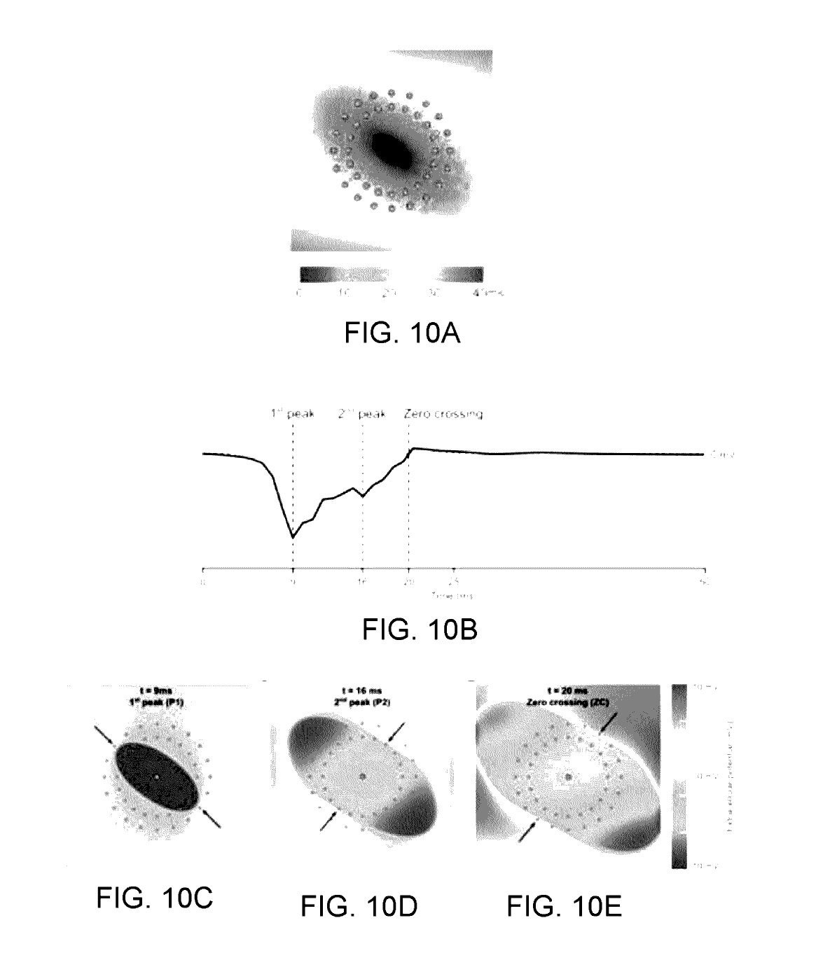

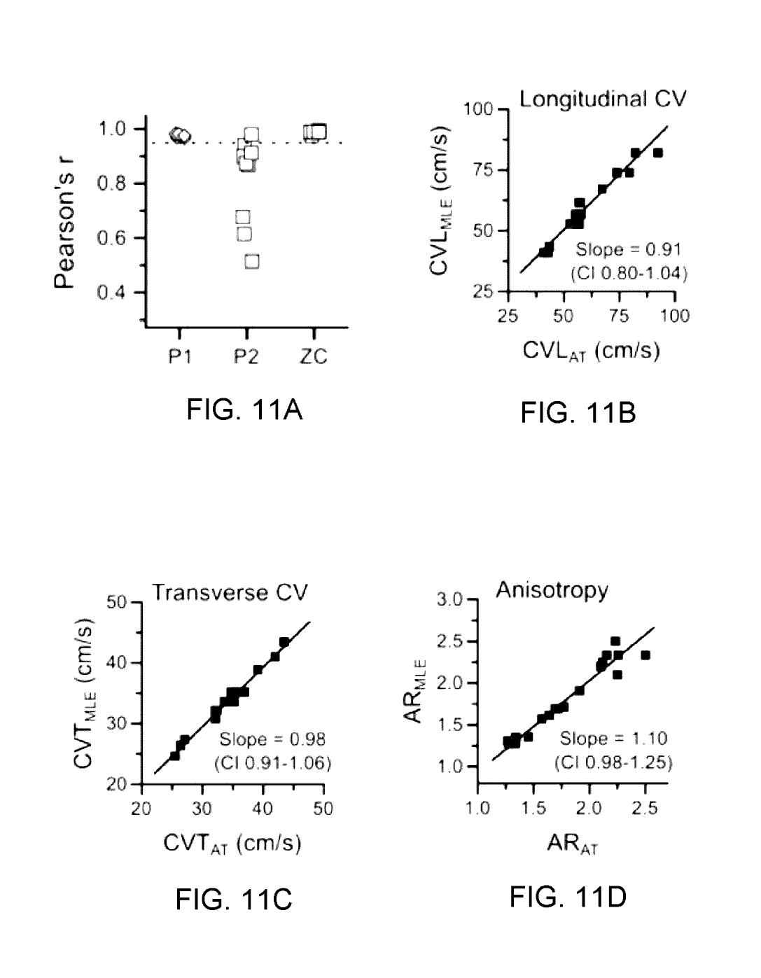

The dual ring array (DRA) configuration was evaluated, and a representative simulated activation time map is shown in FIG. 10A, with the DRA electrode configuration overlaid. The corresponding MLE was calculated using the formula provided above and is shown in FIG. 10B. In contrast to the MLE based on the 8×8 grid configuration, the two peaks were much less distinct. A first major peak can easily be recognized. The distinct peaks were a feature of the heterogeneous coefficients assigned to the electrodes on the 8×8 grid (FIG. 8C). To improve the interpretation of the features of the MLE signal, the temporal evolution of the signal was compared to isopotential maps during the first 25 ms of the simulation. The comparison showed that the location of the major peak (P1) corresponded to the time when the propagating wavefront first passed the outer ring electrodes (FIG. 10C) longitudinally relative to the fiber direction. Similarly, a second peak (P2) coincided with the end of the wavefront passing the last of the inner electrodes (FIG. 10D) transverse (perpendicular) to the fiber orientation. As the end of the wavefront passed the last electrodes on the outer ring, the MLE crossed the isoelectric line (FIG. 10E; zero crossing (ZC)) also along the transverse direction. Identifying the exact location of the wavefront, and thus the distance from the pacing site corresponding to these MLE landmarks, allowed for estimation of the longitudinal CV (from Pb) and transverse CV (from P2 or ZC). A series of 20 simulations were performed in which longitudinal and transverse conductivities were varied to produce a range of conduction velocities. The simulations were repeated with DRA configurations of 10, 15 and 20 electrodes on each ring as well as at three angles of rotation for each DRA configuration. For CVL versus P1, CVT versus P2 and CVT versus ZC, the correlations were quantified for each angle of each DRA configuration, giving a total of 9 correlation coefficients for each combination. FIG. 11A shows the resulting correlation coefficients. The graph demonstrates that P1 and ZC consistently correlated strongly with the traditional measures of CV (r>0.95 for all correlations) while P2 is less reliable. In general, it was more difficult to identify the second peak in the MLEs while the first peak and the zero crossing were easily identified, which is reflected in the variability of the correlation coefficients. For the best performing MLE landmarks, P1 and ZC, the correlations were fairly insensitive to both the number of electrodes and angle of rotation. For both P1 and ZC, a type II linear regression was performed to estimate the potential bias in CV compared to the traditional method of CV estimation (FIGS. 11B and 11C). Compared to CVL and CVT, P1 and ZC respectively showed no significant bias, i.e., the slope of the regression line was not different from 1 (P1, slope=0.91 (CI 0.80-1.04); ZC, slope=0.98 (CI 0.91-1.06)). Additionally, the ratio of P1 to ZC estimates of CV also correlated strongly with CV anisotropy (ratio of CVL to CVT) (r>0.95 for all correlations) and showed no significant bias (slope=1.10 (CI 0.98-1.25), FIG. 11D). Taken together, the simulation results indicated that the DRA based MLE can be used to robustly estimate longitudinal and transverse conduction velocities and the resulting anisotropy from a single signal without the need of assigning activation times to multiple individual UEs.

The electrode array provided an approximation to the surface Laplacian of the potential distribution, φ. Mathematically, this is represented for the continuous function φ (x, y) as

$${∇}^{2} φ = \frac{{\partial }^{2} φ}{\partial {x}^{2}} + \frac{{\partial }^{2} φ}{\partial {y}^{2}}$$

From Taylor series, it was possible to determine discrete approximations to the surface Laplacian as the 3×3 stencil

$$S = \frac{1}{6} ( \begin{matrix}1 & 4 & 1 \\ 4 & - 20 & 4 \\ 1 & 4 & 1\end{matrix} )$$

An approximation to the surface Laplacian can be obtained by considering a 3×3 array of measured potentials φ_{i,j}

$$Φ ( i , j ) = ( \begin{matrix}{φ}_{i - 1 , j - 1} & {φ}_{i , j - 1} & {φ}_{i + 1 , j - 1} \\ {φ}_{i - 1 , j} & {φ}_{i , j} & {φ}_{i + 1 , j} \\ {φ}_{i - 1 , j + 1} & {φ}_{i , j + 1} & {φ}_{i + 1 , j + 1}\end{matrix} )$$

The approximation to the surface Laplacian was obtained from the Frobenius product of two matrices S and Φ(i, j) i.e., the Laplacian at the node (i, j), denoted Lij is given by

Lᵢⱼ=S:Φ(i,j)

where the operator ‘:’ represents the Frobenius product. The following is the long hand formula:

Lᵢⱼ=⅙(φ_{i-1,j-1}+4φ_{i,j-1}+φ_{i+1,j-1}+4φ_{i-1,j}−20φ_{i,j}+4φ_{i-1,j}+φ_{i-1,j+1}+4φ_{i,j+1}+φ_{i+1,j+1})

Finally, the MLE was determined as the average over all possible Laplacians Lij. That is, for an n×n potential electrode array,

$$MLE = \frac{1}{N} {\sum }_{i = 2}^{n - 1} {\sum }_{j = 2}^{n - 1} {L}_{ij}$$

for the Laplacians Lij that exist and N is the total number of Laplacians that exist. For example, for the 8×8 array currently in use, assuming that all 63 recording electrodes perform correctly, then 35 Laplacians can be obtained.

Hence, for the 8×8 electrode array considered herein, the coefficient stensil is given by

$${S}_{MLE} = \frac{1}{216} ( \begin{matrix}1 & 5 & 6 & 6 & 6 & 6 & 5 & 1 \\ 5 & –11 & –6 & –6 & –6 & –6 & –11 & 5 \\ 6 & –6 & 0 & 0 & 0 & 0 & –6 & 6 \\ 6 & –6 & 0 & 0 & 0 & 0 & –6 & 6 \\ 6 & –6 & 0 & 0 & 0 & 0 & –6 & 6 \\ 6 & –6 & 0 & 0 & 0 & 0 & –6 & 6 \\ 5 & –11 & –6 & –6 & –6 & –6 & –11 & 5 \\ 1 & 5 & 6 & 6 & 6 & 6 & 5 & 1\end{matrix} )$$

To evaluate why this is mathematically reasonable, a continuous variation in potential, φ, over some two dimensional domain Ω was considered. The surface Laplacian is then

$$l ( x , y , t ) = {∇}^{2} φ = \frac{{\partial }^{2} φ}{\partial {x}^{2}} + \frac{{\partial }^{2} φ}{\partial {y}^{2}}$$

Averaging the Laplacian over the region Ω was again equivalent to taking the integral over the region, hence

L(t)=∫∫_{Ω}l(x,y,t)dxdy=∫∫_{Ω}∇²φdΩ= [symbol] ∇φ·ndω

which follows from the divergence therein. Here, ∂Ω represents the boundary of the region Ω, n is the outward pointing normal to Ω and dω represents a line element along the boundary ∂Ω. Essentially, the average Laplacian can be obtained from a knowledge of the normal derivatives of the potentials around the boundary of the domain, regardless of the shape of the domain. ∂

Discussion

Example 3 shows that MLE is a method for extracting fundamental features of action potential propagation in cardiac tissue. The experimental data described herein demonstrates that the morphological features of the MLE are closely related to discrete events in the propagation of excitation that occurs following local pacing of the myocardium. The temporal locations of the two primary peaks appear to correlate well with longitudinal and transverse components of conduction. The computer simulations were able to reproduce the experimental data and added the finding that the MLE morphology was dependent on the rotation of recording array with respect to fiber direction. The source of the rotational dependence was traced mathematically to the heterogeneous weighing of the contribution of the electrodes located in the corners of the array. Furthermore, through the simulations a dual-ring electrode configuration was developed and the optimal hallmarks of the MLE were identified to extract measures of conduction. The dual-ring configuration can make the general approach more readily adaptable for clinical use.

The disclosed experimental procedure demonstrated the value of computation models for explicating complex electrophysiological phenomena and their relationship to recording arrays. The computational models utilized clarified the interactions between wavefront propagation and the geometry of the plaque array that produce the characteristic features of the MLE. This ability to visualize and rapidly permute through many possible scenarios was critical to the discovery of the rotational sensitivity of the plaque array, and development of the rotationally invariant DRA. Furthermore, experimental evaluation of the effect of a range of conductivities on the morphological features of the MLE would have been prohibitive.

At its most basic level, the MLE can be interpreted as a summation of electrograms acquired from an array of bipolar electrodes oriented radially around a pacing site (FIGS. 9A and 9B). The relatively simple array geometry and signal processing of the MLE makes it a tool for the characterization of cardiac conduction properties, i.e. CVs and anisotropy of conduction. The conversion of the MLE sampling array from a plaque array to the DRA configuration substantially improves the feasibility of deploying an MLE sensing array in a percutaneous electrophysiology study.

Clinical deployment of the MLE in patients with atrial fibrillation (AF) could facilitate assessment of substrate remodeling and its impact on impulse conduction in the atria. The AF atria are subjected to many remodeling processes that have been shown to impact impulse propagation. Many of these remodeling processes appear to differentially impact the transverse aspect of myocytes to a greater extent than the longitudinal aspect. In light of these observations, robust examination of the anisotropy of conduction can provide valuable insight into the state of AF induced remodeling. The experimental results disclosed herein are based on ventricular recordings from guinea pig ventricles. However, because of the potential for clinical application of this approach in the atria the simulations were run using an atrial myocyte model and a tissue slab with thickness most akin to atrial tissue (3 mm). In spite of this difference, the basic morphology of the MLE was conserved between the experimental and simulation studies.

Described herein is a method in which a novel single electrical signal, the MLE, constructed from a number of simultaneously recorded unipolar electrograms, can be used to quantify both velocity and anisotropy of conduction without the need to assign activation times to individual uni- or bipolar electrograms and without explicit knowledge of the spatial propagation of the wavefront. In consideration of the clinical feasibility, the simplicity of the signal analysis, and the value of robust characterization of cardiac conduction properties, the MLE is a promising addition to current substrate mapping strategies.

Further, Example 3 illustrates the ability of cardiac electrophysiological simulations to help clarify the mechanisms that govern the genesis of electrical signals from the heart. In the case of the MLE, it was shown that the observed peaks were related to the spread of activation across the recording array. Moreover, clarification of the mathematical basis of the MLE acquisition and simulations provided greater insight into the morphology of the MLE. Additionally, it was through permutation of model parameters that the impact of the plaque array's rotational asymmetry was established. Thus, it was possible to conceptualize the DRA for simplified and clinically feasible sampling of the MLE.

The disclosed systems and methods shown the ability of multi-scale modeling to sufficiently replicate cardiac electrophysiological processes in order to examine, interpret, and refine substrate mapping procedures. Consequently, not only does the present disclosure describe methods for substrate mapping, but it is contemplated that the simulations disclosed herein can be used in the development and validation of electrophysiological diagnostics.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Although several embodiments of the invention have been disclosed in the foregoing specification and the following appendix, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. An electrophysiology mapping method comprising:
contacting myocardial tissue with at least three non-collinear electrodes of a plurality of electrodes, wherein the at least three non-collinear electrodes concurrently contact the myocardial tissue;
using a pulse generator to deliver a selected electrical pacing protocol to the at least three non-collinear electrodes, wherein the selected electrical pacing protocol comprises at least one pacing wave;
during each pacing wave of the selected electrical pacing protocol, generating an electrical activation pattern using at least one pair of adjacent electrodes of the at least three non-collinear electrodes and using at least one additional electrode of the plurality of electrodes to detect the activation pattern;
for each pacing wave, using a processor in communication with the plurality of electrodes and the pulse generator to determine a conduction velocity vector associated with the detection of the electrical activation pattern at a corresponding electrode;
using the processor to determine a singular value decomposition of the conduction velocity vectors determined during the electrical pacing protocol; and
determining longitudinal and transverse conduction velocities within the myocardial tissue based upon the singular value decomposition of the conduction velocity vectors.

2. The electrophysiology mapping method of claim 1, further comprising, during each pacing wave of the selected electrical pacing protocol:
using the at least one additional electrode to record an electrogram; and
using the processor to determine an activation time of a corresponding recording electrode based upon each recorded electrogram; and
using the processor to determine the conduction velocity vector associated with the corresponding recording electrode based upon the determined activation time and a known distance between a pacing electrode and the corresponding recording electrode.

3. The electrophysiology mapping method of claim 1, wherein the at least one pacing wave comprises a plurality of pacing waves, and wherein during each pacing wave, a single pair of adjacent electrodes generates a corresponding electrical activation pattern through the myocardial tissue.

4. The electrophysiology mapping method of claim 1, wherein each pacing wave is a depolarization wave.

5. The electrophysiology mapping method of claim 1, wherein each pacing wave has a cycle length ranging from about 100 ms to about 1 s.

6. The electrophysiology mapping method of claim 1, wherein each pacing wave has an amplitude ranging from about 5 mA to about 15 mA.

7. The electrophysiology mapping method of claim 1, wherein each pacing wave has a pulse width ranging from about 1 ms to about 3 ms.

8. The electrophysiology mapping method of claim 1, wherein the at least one additional electrode that detects the electrical activation pattern comprises each of the electrodes of the at least three non-collinear electrodes that is not used to generate the electrical activation pattern.

9. The electrophysiology mapping method of claim 8, wherein the at least one additional electrode that detects the electrical activation pattern further comprises at least one reference electrode that does not contact the myocardial tissue.

10. The electrophysiology mapping method of claim 1, wherein the at least three non-collinear electrodes are supported on a catheter, and wherein the method comprises positioning the catheter within a heart of a subject to contact myocardial tissue within the heart with the at least three non-collinear electrodes.

11. The electrophysiology mapping method of claim 10, wherein the catheter is a loop catheter having a loop portion, and wherein the at least three non-collinear electrodes are secured to the loop portion of the catheter.

12. The electrophysiology mapping method of claim 10, wherein the catheter is a basket electrode catheter having a longitudinal axis and a plurality of splines, wherein each of the at least three non-collinear electrodes is secured to a spline of the basket electrode catheter, wherein the plurality of splines of the catheter are selectively deformable about and between a radially retracted condition and a radially expanded condition, wherein in the radially expanded condition, the splines are compressed such that an intermediate portion of each spline extends radially outwardly relative to the longitudinal axis of the catheter, and wherein the method comprises positioning the splines of the catheter in the radially expanded condition to contact myocardial tissue within the heart with the at least three non-collinear electrodes.

13. The electrophysiology mapping method of claim 12, wherein the plurality of electrodes further comprises a central reference electrode positioned within a central area defined by the plurality of splines of the catheter, and wherein when the plurality of splines are in the radially expanded condition, the central reference electrode is equidistant from the at least three non-collinear electrodes.

14. An electrophysiology mapping system comprising:
a plurality of electrodes, wherein at least three of the electrodes are non-collinear and configured to simultaneously contact myocardial tissue within a heart of a subject;
a pulse generator electrically coupled to the electrodes; and
a processor communicatively coupled to the pulse generator and the electrodes,
wherein the pulse generator is configured to deliver a selected electrical pacing protocol to the electrodes, wherein the selected electrical pacing protocol comprises at least one pacing wave, wherein during each pacing wave of the selected electrical pacing protocol, at least one pair of adjacent electrodes is configured to generate an electrical activation pattern and at least one additional electrode is configured to detect the activation pattern,
wherein, for each pacing wave, the processor is configured to determine a conduction velocity vector associated with the detection of the electrical activation pattern at a corresponding electrode,
wherein the processor is configured to determine a singular value decomposition of the conduction velocity vectors determined during the electrical pacing protocol, and
wherein the processor is configured to determine longitudinal and transverse conduction velocities based upon the singular value decomposition of the conduction velocity vectors.

15. The electrophysiology mapping system of claim 14, further comprising a catheter that supports the plurality of electrodes.

16. The electrophysiology mapping system of claim 15, wherein the catheter is a loop catheter having a loop portion, and wherein the at least three non-collinear electrodes are secured to the loop portion of the catheter.

17. The electrophysiology mapping system of claim 15, wherein the catheter is a basket electrode catheter having a plurality of splines, and wherein each of the at least three non-collinear electrodes is secured to a spline of the basket electrode catheter.

18. The electrophysiology mapping system of claim 17, wherein the catheter has a longitudinal axis, wherein the plurality of splines of the catheter are selectively deformable about and between a radially retracted condition and a radially expanded condition, wherein in the radially expanded condition, the splines are compressed such that an intermediate portion of each spline extends radially outwardly relative to the longitudinal axis of the catheter.

19. The electrophysiology mapping system of claim 18, wherein the plurality of electrodes further comprises a central reference electrode positioned within a central area defined by the plurality of splines of the catheter.

20. The electrophysiology mapping system of claim 19, wherein when the plurality of splines are in the radially expanded condition, the central reference electrode is equidistant from the at least three non-collinear electrodes.
